(19) United States
(12) Patent Application Publication
(10) Pub. No.: US 2026/0207889 A1
(43) Pub. Date: Jul. 23, 2026

(54) Variable Profile Implantable Catheter

(71) Applicant: Bard Peripheral Vascular, Inc., Franklin Lakes, NJ (US)

(72) Inventors: Katelyn Frances Keberle, Tempe, AZ (US); Armando Licon, Tempe, AZ (US); Genevieve Messina, Mesa, AZ (US); Kyle Andrew Thomas, Phoenix, AZ (US); Luke Winterstein, Tempe, AZ (US)

(21) Appl. No.: 19/135,079

(22) PCT Filed: Dec. 6, 2022
(86) PCT No.: PCT/US2022/052005

§ 371 Date: Jun. 3, 2025

(51) Int. Cl. A61M 25/00; A61M 25/01; A61M 39/02

(52) U.S. Cl. CPC A61M 25/005; A61M 25/0023; A61M 25/0102; A61M 2025/0024; A61M 2025/0025; A61M 2025/0059; A61M 39/0247; A61M 2210/12

(57) ABSTRACT

Catheters having an expanded configuration define a catheter lumen to provide fluid communication during fluid delivery. However, in between fluid delivery events, the catheter occupies a cross-sectional area within a vessel reducing the patency thereof and increasing the risk of occlusion formation. Embodiments include a catheter tube configured to collapse radially inward in between fluid delivery events to reduce the impact on the patency of the vessel and mitigate thrombosis formation. A fluid pressure within the catheter tube can transition the catheter to the expanded configuration. Catheter systems can further include an inflatable stiffening member configured to impart rigidity on the catheter tube to facilitate placement. Further, the catheter tube can include a reinforcement member configured to provide longitudinal strength while still allowing radial transition to the collapsed configuration. The reinforcement member can be inflatable to further reduce a cross-sectional area in between fluid delivery events.

### BACKGROUND

Medium to long term intravascular catheters include a portion of the catheter residing within the vasculature for an extended period of time. During use, the catheter defines a catheter lumen through which fluids can be delivered to the vasculature of a patient. Further, such catheters can include reinforcement members to modify the rigidity of the catheter and prevent “whipping” of a tip portion during fluid delivery, which can damage the vessel.

In between uses, the presence of the catheter within the vessel can affect a blood flow therethrough. The cross-sectional area of the catheter can reduce the overall cross-sectional area (patency) of the vessel and, as such, increase a fluid pressure on the wall of the vessel. The reduced cross-sectional area of the vessel, or reduced linear distance between the wall of the vessel and the outer surface of the catheter, can increase the chance of thrombosis formation which can further reduce the patency of the vessel, creating blood clots, and cause complications to the patient.

Current standards of care weigh the aforementioned problems against the trauma and discomfort of removing the catheter between uses. What is needed therefore is a medium to long term intravascular catheter that can remain in place between fluid delivery events but can also provide a reduced cross-sectional profile in between fluid delivery events.

### SUMMARY

Briefly summarized, embodiments disclosed herein are directed to a collapsible catheter that transitions between an expanded configuration and a collapsed configuration. In the expanded configuration the catheter defines an increased cross-sectional area to the catheter lumen, and to the catheter as a whole, for the delivery of fluids therethrough. In the collapsed configuration the catheter defines a decreased cross-sectional area to the catheter lumen, and to the catheter as a whole, to reduce the impact on the patency of the vessel in between fluid delivery events. The catheter system can further include a stiffening member to facilitate placement of the catheter within the vasculature. Embodiments include an inflatable stiffening member that can apply a radial outward pressure on the catheter and impart more rigid mechanical properties during placement within a vasculature.

Embodiments also include a catheter having a reinforcement member formed as part of the catheter wall and configured to modify the rigidity of the catheter while still allowing the catheter to transition between the expanded configuration and the collapsed configuration. Embodiments include an inflatable reinforcement member that transitions between an inflated state and a deflated state. In the inflated state, the reinforcement member increases the rigidity of the catheter and/or facilitates transitioning the catheter between the collapsed configuration and the expanded configuration. In the deflated state, the reinforcement member provides a reduced cross-sectional area of the catheter.

Disclosed herein is a catheter system including, a catheter tube defining a lumen and transitionable between an expanded configuration defining a relatively larger cross-sectional area, and a collapsed configuration defining a relatively smaller cross-sectional area, the catheter tube including a longitudinally extending reinforcement member, and a stiffening member, disposed within the lumen and transitionable between an inflated configuration and a deflated configuration, wherein the stiffening member in the inflated configuration is designed to apply a radial outward force against an inner wall of the catheter lumen to mitigate kinking and/or columnar collapse.

In some embodiments, the stiffening member in the inflated configuration transitions the catheter tube to the expanded configuration.

In some embodiments, the stiffening member in the inflated configuration imparts an increased rigidity on the catheter tube.

In some embodiments, the stiffening member extends along a longitudinal length of the catheter lumen between a proximal end and a distal end.

In some embodiments, the catheter tube is biased towards a collapsed configuration. For example, the catheter tube may be first formed, i.e. manufactured, in a shape having the collapsed configuration (crescent or oval cross-sectional shape), and may be formed of a sufficiently resilient material to be expanded under pressure to the expanded configuration.

In some embodiments, the catheter tube is biased towards an expanded configuration. For example, the catheter tube may be first formed, i.e. manufactured, in a shape having the expanded configuration (substantially circular cross-sectional shape), and may be of a sufficiently resilient material to be collapsed under pressure to the collapsed configuration.

In some embodiments, the catheter tube further includes one or more living hinges extending linearly along a longitudinal axis of the catheter tube and configured to facilitate transitioning the catheter tube between the expanded configuration and the collapsed configuration.

In some embodiments, the reinforcement member extends linearly along a longitudinal axis over at least a portion of the catheter tube.

In some embodiments, the reinforcement member extends helically along a longitudinal axis over at least a portion of the catheter tube.

In some embodiments, at least a portion of the reinforcement member is coupled to an outer surface of the catheter tube.

In some embodiments, at least a portion of the reinforcement member extends through a wall of the catheter tube.

In some embodiments, the reinforcement member is formed integrally with the catheter tube.

In some embodiments, the reinforcement member is co-extruded with the catheter tube.

In some embodiments, the catheter tube in the collapsed configuration defines a crescent shaped cross-sectional shape.

In some embodiments, the catheter tube in the collapsed configuration defines an oval cross-sectional shape.

In some embodiments, the catheter tube and the reinforcement member may be formed of a first material. Optionally, the reinforcement member may be formed integrally with the catheter tube.

In some embodiments, the catheter tube is formed of the first material and wherein the reinforcement member is formed of a second material having an increased rigidity relative to the first material.

In some embodiments, the reinforcement member defines a lumen and is configured to receive an inflation fluid therein to transition the reinforcement member between a deflated configuration and an inflated configuration.

In some embodiments, the reinforcement member in the inflated configuration imparts an increased rigidity on the catheter tube relative to the reinforcement member in the deflated configuration.

Also disclosed is a method of placing a catheter including, transitioning a stiffening member from a deflated configuration to an inflated configuration, the stiffening member disposed within a lumen of a catheter tube, transitioning the catheter tube to an expanded configuration by the stiffening member applying a radial outward force on an inner surface of the lumen of the catheter tube and imparting an increased rigidity on the catheter tube, advancing the catheter tube intravascularly to a target location, transitioning the stiffening member from the inflated configuration to the deflated configuration, and transitioning the catheter tube from the expanded configuration to a collapsed configuration.

In some embodiments, the method further includes applying a fluid flow to the lumen of the catheter tube to transition the catheter tube from the collapsed configuration to the expanded configuration.

In some embodiments, in the collapsed configuration, a first portion of the inner surface of the catheter lumen contacts a second portion of the inner surface of the catheter lumen, the first portion being disposed opposite the second portion across a central axis of the catheter lumen.

In some embodiments, the catheter tube further includes a reinforcement member extending longitudinally along the catheter tube.

In some embodiments, the method further includes inflating the reinforcement member to transition the reinforcement member from a deflated state to an inflated state.

In some embodiments, the reinforcement member in the inflated state imparts rigid mechanical properties on the catheter tube.

In some embodiments, the reinforcement member in the inflated state transitions the catheter tube from a collapsed configuration to an expanded configuration.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A shows a perspective view of a catheter system in an expanded configuration, in accordance with embodiments disclosed herein.

FIG. 1B shows a perspective view of a catheter system in a collapsed configuration, in accordance with embodiments disclosed herein.

FIG. 2A shows close up detail of a distal portion of a catheter system in an expanded configuration disposed within a vessel and including a linear support, in accordance with embodiments disclosed herein.

FIG. 2B shows close up detail of a distal portion of a catheter system in a collapsed configuration disposed within a vessel and including a linear support, in accordance with embodiments disclosed herein.

FIG. 3 shows a catheter system in an expanded configuration and including a helical support, in accordance with embodiments disclosed herein.

FIG. 4A shows a longitudinal cross-sectional view of a catheter tube including an inflatable stiffening member in an inflated configuration, in accordance with embodiments disclosed herein.

FIG. 4B shows a longitudinal cross-sectional view of a catheter tube including an inflatable stiffening member in a deflated configuration, in accordance with embodiments disclosed herein.

FIG. 5A shows a lateral cross-sectional view of a catheter including an inflatable reinforcement member, or support, in an expanded configuration, in accordance with embodiments disclosed herein.

FIG. 5B shows a lateral cross-sectional view of a catheter including an inflatable reinforcement member, or support, in a collapsed configuration, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

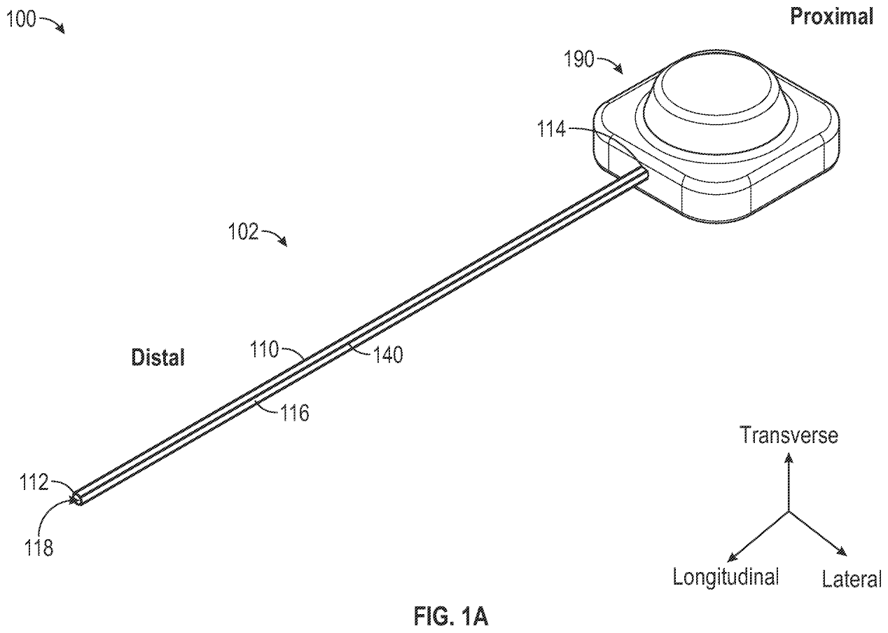

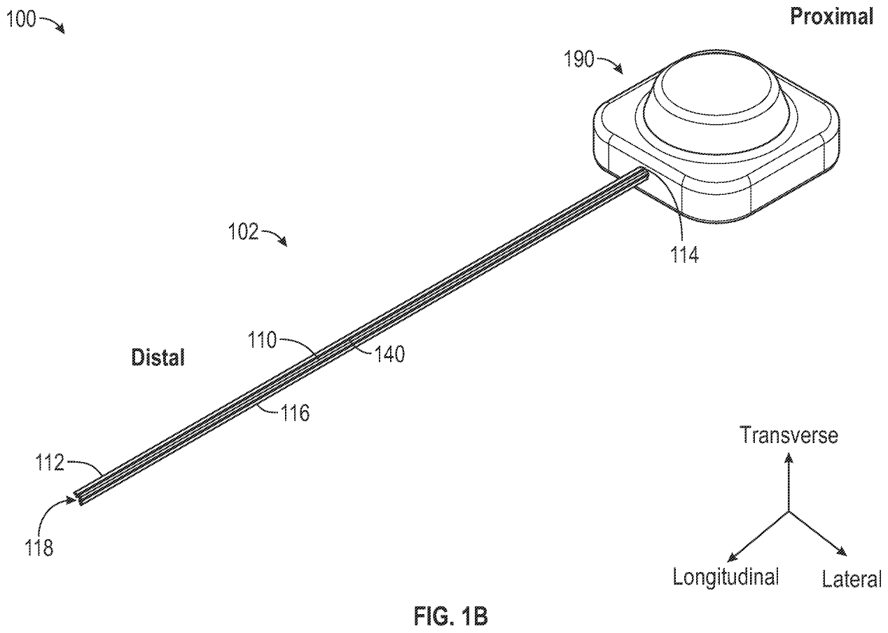

To assist in the description of embodiments described herein, as shown in FIG. 1A, a longitudinal axis extends substantially parallel to an axial length of the catheter. A lateral axis extends normal to the longitudinal axis, and a transverse axis extends normal to both the longitudinal and lateral axes.

Unless defined otherwise, all technical and scientific terms used herein have the same meaning as commonly understood by those of ordinary skill in the art.

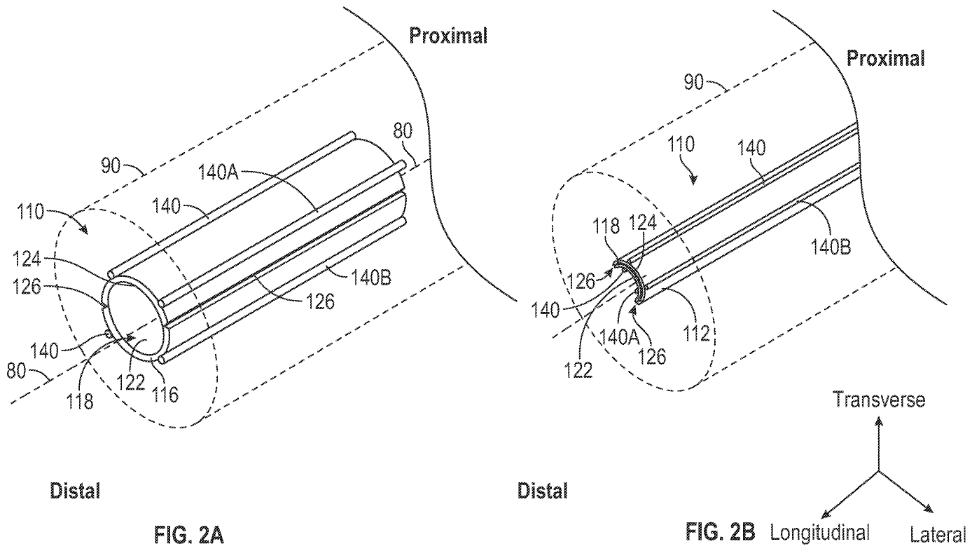

FIGS. 1A-2B show details of a catheter system 100 generally including a catheter 102 having a catheter tube 110 extending longitudinally between a distal tip 112 and a proximal end 114. Exemplary catheters 102 include a peripherally inserted central catheter (PICC), central venous catheter (CVC), midline catheter, single lumen catheter, dual lumen catheter, multi-lumen catheter, dialysis catheter, or the like. In an embodiment, as shown in FIG. 1A, the proximal end 114 of the catheter tube 110 can be in fluid communication with an access device 190 such as a subcutaneous access port, or the like. However, the port is not intended to be limiting and, in an embodiment, the proximal end 114 of the catheter tube 110 can also be in fluid communication with one or more of a catheter hub, bifurcation, one or more extension legs, fluid connectors, luer locks, spin nuts, combinations thereof, or the like.

The catheter tube 110 includes a wall 116 defining a catheter lumen 118. In an embodiment, the wall 116 of the catheter tube 110 is formed of a first material including one or more of a plastic, polymer, elastomer, rubber, silicone rubber, polytetrafluoroethylene (PTFE), expanded polytetrafluoroethylene (ePTFE), nylon, polyethylene terephthalate (PET), and/or polyurethane (PU), combinations thereof, or the like. In an embodiment, the first material defines a relatively softer, lower durometer, and/or relatively more flexible mechanical properties. Advantageously, the first material of the catheter tube 110 facilitates negotiating tortuous vascular pathways. In an embodiment, the catheter 102 or portions thereof, e.g. catheter tube 110, can be formed by extrusion, blow molding and/or other suitable manufacturing techniques.

In an embodiment, the catheter tube 110 is configured to transition between an expanded configuration (FIG. 1A) and a collapsed configuration (FIG. 1B). As shown in FIGS. 1A and 2A, in the expanded configuration the catheter tube 110, and the catheter lumen 118, defines a relatively larger lateral cross-sectional area. In the expanded configuration, the catheter lumen 118 defines a substantially circular cross-sectional shape, configured to provide an increased rate of fluid flow therethrough.

As shown in FIGS. 1B and 2B, in the collapsed configuration the catheter tube 110, and the catheter lumen 118, defines a relatively smaller lateral cross-sectional area. As such, the catheter tube 110 occupies a relatively smaller cross-sectional area of the vessel 90. In an embodiment, in the collapsed configuration, a first portion 122 of the inner surface of the catheter lumen 118 contacts a second portion 124 of the inner surface of the catheter lumen 118, the second portion 124 disposed opposite the first portion 122 across a central longitudinal axis 80 of the catheter lumen 118.

In an embodiment, in the expanded configuration, the first portion 122 and the second portion 124 of the catheter tube 110 both define a concave inner surface of the catheter lumen 118. In the collapsed configuration, the first portion 122 of the catheter tube 110 inverts to define a convex inner surface of the catheter lumen 118 and contact a concave inner surface of the second portion 124. Alternatively, in the collapsed configuration, the second portion 124 of the catheter tube 110 inverts to define a convex inner surface of the catheter lumen 118 and contact a concave inner surface of the first portion 122. As such, the collapsed configuration defines a semi-circular, or crescent shaped, cross-sectional shape.

In an embodiment, the catheter tube 110 includes a living hinge 126 such as a score line, groove, laser cut line, or the like extending longitudinally. The living hinge 126 facilitates folding of the catheter wall 116 therealong to allow the catheter tube 110 to transition between the expanded configuration and the collapsed configuration along a predefined axis, e.g. transverse, lateral, or at an angle therebetween. The living hinge 126 may optionally ensure that the catheter tube collapses and expands in a predefined, repeatable, and predictable manner each time that the catheter tube transitions between the expanded configuration and the collapsed configuration.

Advantageously, the semi-circular, or crescent shaped, cross-sectional shape of the collapsed configuration allows the catheter tube 110 to collapse radially while providing longitudinal structural rigidity. The longitudinal structural rigidity mitigates kinking of the catheter when an axial (longitudinal) force is applied. Further, the longitudinal structural rigidity mitigates the distal tip 112 from whipping laterally or transversely back and forth within the vessel 90 as a blood flow flows over the outer surface of the catheter tube 110. The whipping action of the catheter tube 110 can cause damage to an inner wall of the vessel. Further, the crescent cross-sectional shape can mitigate twisting of the catheter about the longitudinal axis, when the catheter tube 110 is in the collapsed configuration. The twisting action can prevent the catheter tube 110 from transitioning to the expanded state and prevent a fluid flow through the catheter lumen 118.

In an embodiment, the catheter tube 110 further includes a reinforcement member, or support 140. The reinforcement member, or support 140, may be disposed in a wall 116 of the catheter tube 110, on an outer surface of the catheter 110, or a combination thereof. The support 140 extends linearly along a longitudinal axis, along at least a portion of the catheter tube 110, or along the entire length of the catheter tube 110. In an embodiment, the support 140 is formed of the first material of the catheter tube 110 and defines a rib extending longitudinally. In an embodiment, the catheter tube 110 and the support 140 can be formed integrally. In an embodiment, the support 140 can be co-extruded with the catheter tube 110. Having the support 140 made of the same material as the catheter tube 110, formed integrally with the catheter tube 110, and/or coextruded with the catheter tube 110 may optionally make it easier to manufacture the catheter tube, e.g. by allowing the support 140 and catheter tube 110 to be formed simultaneously in a single manufacturing step.

In an embodiment, the support 140 is formed of a second material different from that of the first material. The second material can include a plastic, polymer, elastomer, rubber, silicone rubber, polytetrafluoroethylene (PTFE), expanded polytetrafluoroethylene (ePTFE), metal, alloy, and/or composite, combinations thereof, or the like. In an embodiment, the second material defines a relatively harder, more rigid, higher durometer, and/or relatively less flexible mechanical properties relative to the first material. In an embodiment, the support 140 varies in the mechanical properties (e.g. rigidity, flexibility, etc.) along a longitudinal length thereof. In an embodiment, the support 140 varies in composition between the first material and the second material to vary the mechanical properties (e.g. rigidity, flexibility, etc.) along a longitudinal length thereof. As will be appreciated, one or more third materials, different from both the first material and the second material, can also be included in the support 140.

Advantageously, the support 140 is configured to allow radial collapse of the catheter (e.g. transitioning between the collapsed configuration and the expanded configuration), while also providing increased longitudinal rigidity along at least a portion of the catheter tube 110. As such, the support 140 prevents kinking and/or whipping of the catheter tube 110 within the vessel 90, as described herein. For example, kinking can occur when a longitudinal axial force is applied. Further, a whipping action of the distal tip 112 of the catheter tube 110 can occur either due to fluid pressure passing through the catheter lumen 118, and/or from a blood flow passing over the outer surface of the catheter tube 110. As noted, the whipping action can cause trauma to the inner surface of the vessel 90.

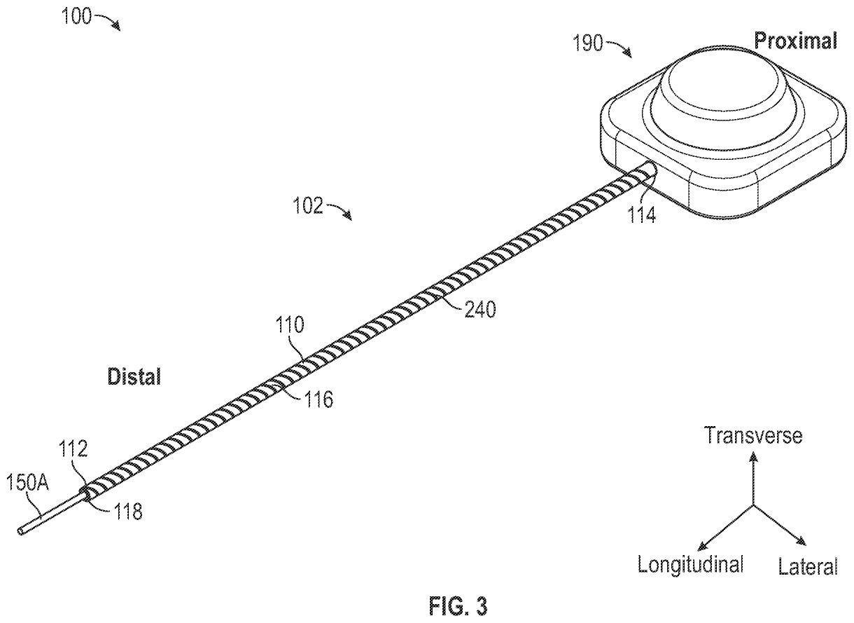

In an embodiment, as shown in FIG. 3, the support 240 extends helically along the longitudinal axis and about an outer surface of at least a portion of the catheter tube 110 and/or through at least a portion of a wall 116 of the catheter tube 110, as described herein. In an embodiment, the helical support 240 can be formed of the first material, the second material, one or more third materials, or a combination thereof, as described herein. In an embodiment, the helical support 240 can be formed integrally with the catheter tube 110 or formed separately and co-extruded with the catheter tube 110, and/or coupled with the catheter tube 110, as described herein.

It is important to note that the longitudinal linear support 140 or the helical support 240 modifies the mechanical properties (e.g. rigidity) of the catheter tube 110 while still being flexible enough to allow the catheter tube 110 to transition between the expanded configuration and the collapsed configuration, as described herein. For example, the linear support 140 provides columnar strength while still allowing the catheter tube 110 to hinge along an axis, e.g. along living hinge 126, disposed between a first longitudinal support 140A and a second longitudinal support 140B to allow the catheter tube 110 to transition between the expanded configuration and the collapsed configuration.

As another example, the helical support 240 can be flexible enough to allow the catheter tube to flex, e.g. along living hinge 126 and transition to the collapsed configuration. In an embodiment, the helical support 240 is configured to provide columnar strength but still be flexible enough to allow the pressure of the blood within the vessel 90 to transition the catheter tube 110 to the collapsed configuration. In an embodiment, the helical support 240 can include one or more living hinges aligned with the living hinge 126 of the catheter body and configured to facilitate transitioning the catheter 110 between the expanded configuration and the collapsed configuration, as described herein.

In an embodiment, the catheter tube 110 is biased towards the collapsed configuration and a fluid pressure from within the catheter lumen 118 transitions the catheter tube 110 to the expanded configuration. For example, the catheter tube 110 can be formed (i.e. manufactured) in a crescent cross-sectional shape. In use, the flexibility of the first material of the catheter tube 110 allows the catheter tube 110 to transition to the expanded configuration, as described herein.

In an embodiment, the catheter tube 110 is biased towards the expanded configuration and a fluid pressure from the blood within the vessel 90 applies a radially inward pressure to an outer surface of the catheter tube 110 and transitions the catheter tube 110 to the collapsed configuration. For example, the catheter tube 110 can be formed (i.e. manufactured) in a substantially circular cross-sectional shape. In use, the flexibility of the first material of the catheter tube 110 allows the catheter tube 110 to transition to the collapsed configuration, as described herein.

In an embodiment, the catheter system 100 further includes a stiffening member 150 disposed within the catheter lumen 118. The stiffening member 150 can provide columnar strength to the catheter tube 110 during placement within the vasculature. In an embodiment, the stiffening member 150 can be a solid stiffening member 150A such as a stylet, guidewire, or similar elongate medical device. In an embodiment, the solid stiffening member 150A maintains the catheter tube 110 in the expanded configuration. In an embodiment, the solid stiffening member 150A can be thin enough to be disposed within the catheter lumen 118 while the catheter tube 110 is in the collapsed configuration or in a transitional (partially collapsed) configuration between that of the expended and (fully) collapsed configurations. In an embodiment, the stiffening member 150 is advanced to a target location within the vasculature. The catheter 102 is then be advanced over the stiffening member 150 in either of the expanded or collapsed configurations into the vasculature to the target location. The stiffening member 150 is then withdrawn proximally from the catheter lumen 118.

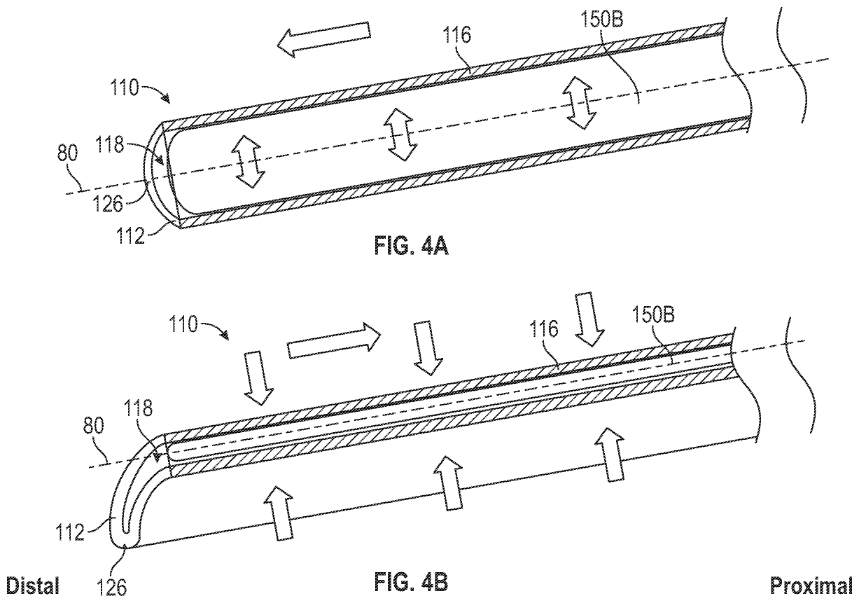

In an embodiment, as shown in FIGS. 4A-4B, the stiffening member 150 is an inflatable stiffening member 150B such as an elongate inflatable balloon disposed within the catheter lumen 118 and can be inflated with an inflation fluid (gas or liquid) to provide a rigid structure. For example, as shown in FIG. 4A, an inflatable stiffening member 150B is disposed within the catheter lumen 118, e.g. in a deflated configuration, and inflated to expand radially outwards. The radially outward pressure on the inner surface of the catheter lumen 118 transitions the catheter tube 110 to the expanded configuration. In the expanded configuration the inflatable stiffening member 150B applies radially outward pressure to impart rigid mechanical properties on the catheter tube 110. The catheter tube 110 and inflatable stiffening member 150B assembly is then advanced longitudinally into the vasculature while preventing kinking or collapse of the catheter tube 110 as the longitudinal force is applied.

In an embodiment, inflating the inflatable stiffening member 150B causes the inflatable stiffening member 150B to expand both radially outwards and longitudinally through the catheter lumen 118. As such, the inflatable stiffening member 150B advances through the catheter lumen 118, transitioning the catheter tube 110 to the expanded configuration, and/or imparting rigid mechanical properties on the catheter tube 110, as described herein.

In an embodiment, the catheter system 100 includes a first (solid) stiffening member 150A, e.g. guidewire, and a second (inflatable) stiffening member 150B, each extending through the catheter lumen 118. In an embodiment, the solid stiffening member 150A and the inflatable stiffening member 150B extend side by side through the catheter lumen 118. In an embodiment, the solid stiffening member 150A and the inflatable stiffening member 150B extend coaxially through the catheter lumen 118. For example, the inflatable stiffening member 150B can define a toroidal cross-sectional shape with the solid stiffening member 150A extending therethrough. As such, the inflatable stiffening member 150B transitions the catheter tube 110 to the expanded configuration, and/or imparts rigid mechanical properties on the catheter tube 110, as described herein. The catheter tube 110 and inflated inflatable stiffening member 150B assembly is then advanced over the solid stiffening member 150A to a target location.

As shown in FIG. 4B, once the distal tip 112 of the catheter 102 is placed at a target location, the inflatable stiffening member 150B can be deflated to reduce any radial outward pressure on the catheter tube 110. With the inflatable stiffening member 150B in the deflated state, the catheter tube 110 resumes relatively more flexible mechanical properties. With the inflatable stiffening member 150B in the deflated state, the catheter tube 110 transitions to a collapsed configuration. The stiffening member 150, e.g. one or both of the solid stiffening member 150A and the inflatable stiffening member 150B are withdrawn proximally from the catheter lumen 118.

In an embodiment, the inflatable stiffening member 150B is advanced through the catheter lumen 118 in the deflated state before being transitioned to the inflated state to impart rigid properties to the catheter tube 110. Similarly, the inflatable stiffening member 150B can be deflated prior to being withdrawn from the catheter lumen 118. Advantageously, this prevents friction or drag on the catheter tube 110 as the stiffening member 150B is advanced or withdrawn from the catheter lumen 118.

In an embodiment, the inflatable stiffening member 150B can be inflated by an active or passive fluid pump in fluid communication therewith. In an embodiment, the inflatable stiffening member 150B is in fluid communication with the access device 190, port, catheter hub, extension leg, syringe, combinations thereof, or the like. Accessing the port 190 and administering a fluid to the port 190 can provide an inflation fluid to the inflatable stiffening member 150B,

In an embodiment, the inflatable stiffening member 150B is in fluid communication with a second access device, different from the first access device 190. Accessing the second access device and providing an inflation fluid thereto inflates the inflatable stiffening member 150B, optionally using a second fluid, different from a fluid provided to the catheter lumen 118. This may optionally allow for greater control over the amount of inflation fluid delivered to the inflatable stiffening member 150B to control the amount of rigidity imparted on the catheter tube 110. As will be appreciated the second access port is exemplary, and in an embodiment, a catheter hub, extension leg, etc. extending from the proximal end of the catheter tube is in fluid communication with the inflatable stiffening member 150B and configured to provide an inflation fluid thereto. In an embodiment, the inflatable stiffening member 150B is a separate structure from that of the catheter tube 110. In an embodiment, the inflatable stiffening member 150B is formed integrally with the catheter tube 110, or coupled with in interior surface of the catheter lumen 118 and remains in place with the catheter tube 110. As such, in the deflated state, the inflatable stiffening member 150B has a reduced impact on the patency of the catheter lumen 118 when a fluid is administered thereto.

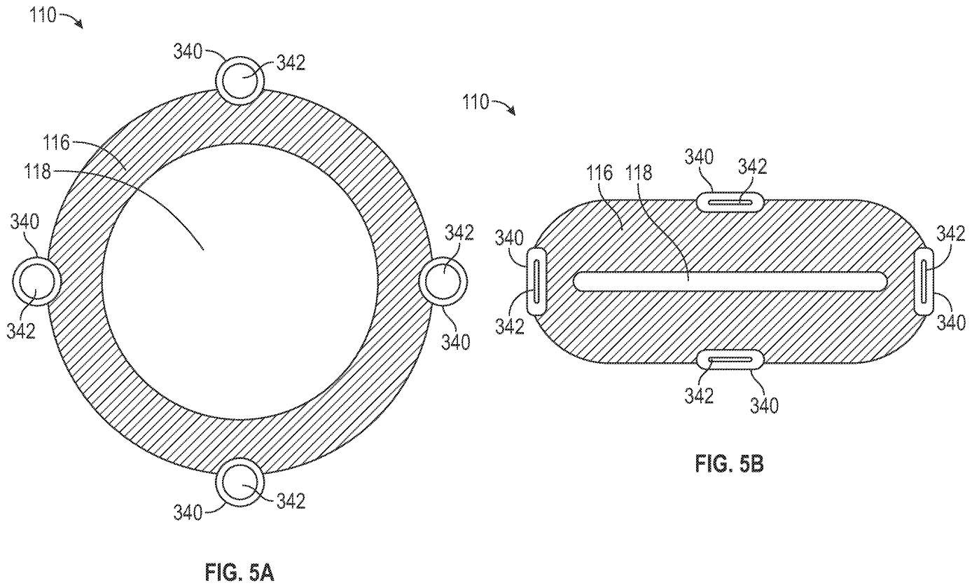

In an embodiment, as shown in FIGS. 5A-5B, the catheter tube 110 can transition to a collapsed configuration including a substantially oblong cross-sectional shape (FIG. 5B). In an embodiment, the catheter system 100 includes an inflatable reinforcement member, or inflatable support 340. The inflatable support 340 defines a support lumen 342 and is filled with an inflation fluid such as a liquid or a gas. FIG. 5A shows the inflatable support 340 in the inflated state. FIG. 5B shows the inflatable support 340 in the deflated state. In the inflated state, a fluid pressure within the lumen 342 of the inflatable support 340 transitions the inflatable support 340 to a relatively more rigid configuration and in turn imparts rigidity to the catheter tube 110. In contrast, in the deflated state, the inflatable support 340 and in turn the catheter tube 110 displays relatively more flexible properties.

In the inflated state the inflatable support 340 includes a relatively high pressure of fluid disposed within the support lumen 342, relative to ambient air pressure. In an embodiment, the deflated state includes a relatively low pressure, relative to the inflated state. In an embodiment, the deflated state includes an ambient fluid pressure. In an embodiment, the deflated state has a volume of inflation fluid removed from the inflatable support lumen 342. In an embodiment, as shown in FIG. 5B, the inflatable supports 340 in the deflated state displays a reduced cross-sectional area relative to the inflated state. Advantageously, this further reduces the overall cross-sectional area of the catheter tube 110 when in the collapsed configuration, further reducing the impact on the patency of the vessel 90.

In an embodiment, the inflatable support 340 can be inflated by an active or passive fluid pump in fluid communication with the inflatable support 340. In an embodiment, the inflatable support 340 is in fluid communication with the subcutaneous port 190. Accessing the port 190 and administering a fluid to the port 190 can both provide a fluid to the catheter lumen 118 and also provide the fluid to the inflatable support lumen 342, inflating the inflatable support 340 with the same fluid. This may optionally provide a simple manner of simultaneously expanding the catheter tube to the expanded configuration and delivering a desired fluid through the lumen of the catheter tube, e.g. using a single connection. In an embodiment, the inflatable support 340 is in fluid communication with a second access device, different from the access device 190. Accessing the second access device and providing an inflation fluid thereto inflates the inflatable support 340, optionally using a second fluid, different from a fluid provided to the catheter lumen. This may optionally allow for greater control over the amount of inflation fluid delivered to the inflatable support 340 to control the amount of rigidity imparted on the catheter tube 110. Further, the amount of inflation fluid (volume/flow rate, etc.) delivered to the inflatable support 340 can be independent of the amount fluid (volume/flow rate, etc.) delivered to the patient via the catheter lumen 118. As will be appreciated the second access port is exemplary, and in an embodiment, a catheter hub, extension leg, etc. extending from the proximal end of the catheter tube is in fluid communication with the inflatable support 340 and configured to provide an inflation fluid thereto.

As described herein, one or more inflatable supports 340 extends longitudinally or helically about the catheter tube 110. In an embodiment, inflating/deflating the inflatable support 340 between the inflated state and the deflated state transitions the catheter tube 110 between the expanded configuration and the collapsed configuration. For example, inflating the inflatable support 340 transitions the catheter tube 110 to the expanded configuration. Similarly, transitioning the inflatable support 340 to the deflated state urges the catheter tube 110 to the collapsed configuration.

Advantageously, the catheter tube 110 in the collapsed configuration provides a smaller overall surface area on which biofilms, fibrin, thrombosis, or similar occlusions can develop. Further, transitioning the catheter tube 110 from the collapsed configuration to the expanded configuration can dislodge any such occlusions that may have formed on the outer surface of the catheter, at a distal tip 112 of the catheter, and/or within the catheter lumen 118 in between fluid delivery events, restoring patency to the catheter lumen 118 and extending the life of the catheter system 100.

In an embodiment, the catheter system 100 further includes an active pressure control system configured to control a fluid pressure within the catheter lumen 118. The active pressure control system maintains a fluid pressure within the catheter lumen 118 sufficient to transition the catheter tube 110 to, and optionally maintain, one of the expanded configuration or the collapsed configuration.

In an embodiment, a distal tip 112 of the catheter 110 includes a valve, for example a Groshong valve, or the like, and configured to control a fluid flow through the distal tip 112 of the catheter lumen 118. Advantageously, the valve disposed at the distal tip 112 is configured to control a fluid pressure within the catheter lumen 118 and as such controls the fluid pressure within the catheter lumen 118 that is required to transition the catheter tube 110 to the expanded configuration. Advantageously, the valve can be tuned to a more specific range of pressures required to transition the catheter tube 110 between the expanded and collapsed configuration. Advantageously, the valve can also mitigate the formation of a fibrin sheath or similar occlusion at the distal tip 112. In an embodiment, the portion of the catheter tube 110 including the valve can transition between the expanded configuration and the collapsed configuration to provide a reduced cross-sectional shape, as described herein, without affecting the functioning of the valve.

In an embodiment, a distal tip 112 of the catheter 110 includes a “non-dynamic” portion of the catheter tube 110. Worded differently, a first section of the catheter tube 110 extending from a point disposed proximally of the distal tip 112 and extending proximally, can transition between the collapsed configuration and the expanded configuration as described herein. As such, a second section disposed distally of the first section can be configured to remain in the expanded configuration. The second section can maintain a fluid lock such as a saline and/or heparin fluid lock within the lumen of the second section and can mitigate the formation of a fibrin sheath or similar occlusion at the distal tip 112.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A catheter system, comprising:
a catheter tube extending between a proximal end and a distal tip, wherein the proximal end is external to a vasculature in use, and wherein the distal tip is internal to the vasculature in use, the catheter tube defining a lumen communicating with a distal opening at the distal tip, the catheter tube transitioning between an expanded configuration defining a relatively larger cross-sectional area, and a collapsed configuration defining a relatively smaller cross-sectional area, the catheter tube including a longitudinally extending reinforcement member extending between the proximal end and the distal tip; and
a stiffening member, disposed within the lumen and transitionable between an inflated configuration and a deflated configuration, wherein the stiffening member in the inflated configuration is designed to apply a radial outward force against an inner wall of the catheter lumen to mitigate kinking or columnar collapse.

2. The catheter system according to claim 1, wherein the stiffening member in the inflated configuration transitions the catheter tube to the expanded configuration.

3. The catheter system according to claim 1, wherein the stiffening member in the inflated configuration imparts an increased rigidity on the catheter tube.

4. The catheter system according to claim 1, wherein the stiffening member extends along a longitudinal length of the catheter lumen between a proximal end and a distal end.

5. The catheter system according to claim 1, wherein the catheter tube is biased towards a collapsed configuration.

6. The catheter system according to claim 1, wherein the catheter tube is biased towards an expanded configuration.

7. The catheter system according to claim 1, wherein the reinforcement member extends linearly along a longitudinal axis over at least a portion of the catheter tube.

8. The catheter system according to claim 1, wherein the reinforcement member extends helically along a longitudinal axis over at least a portion of the catheter tube.

9. The catheter system according to claim 1, wherein at least a portion of the reinforcement member is coupled to an outer surface of the catheter tube.

10. The catheter system according to claim 1, wherein at least a portion of the reinforcement member extends through a wall of the catheter tube.

11. The catheter system according to claim 1, wherein the catheter tube in the collapsed configuration defines a crescent shaped cross-sectional shape.

12. The catheter system according to claim 1, wherein the catheter tube in the collapsed configuration defines an oval cross-sectional shape.

13. The catheter system according to claim 1, wherein the catheter tube is formed of a first material and wherein the reinforcement member is formed of a second material having an increased rigidity relative to the first material.

14. The catheter system according to claim 1, wherein the reinforcement member defines a lumen and is configured to receive an inflation fluid therein to transition the reinforcement member between a deflated configuration and an inflated configuration.

15. The catheter system according to claim 14, wherein the reinforcement member in the inflated configuration imparts an increased rigidity on the catheter tube relative to the reinforcement member in the deflated configuration.

16. A method of placing a catheter, comprising:
transitioning a stiffening member from a deflated configuration to an inflated configuration, the stiffening member disposed within a lumen of a catheter tube, the catheter tube extending between a proximal end and a distal tip, the lumen communicating with a distal opening at the distal tip;
transitioning the catheter tube to an expanded configuration by the stiffening member applying a radial outward force on an inner surface of the lumen of the catheter tube and imparting an increased rigidity on the catheter tube;
advancing the distal tip of the catheter tube intravascularly to a target location, wherein the proximal end of the catheter tube remains external to a vasculature;
transitioning the stiffening member from the inflated configuration to the deflated configuration; and
transitioning the catheter tube from the expanded configuration to a collapsed configuration.

17. The method according to claim 16, further including applying a fluid flow to the lumen of the catheter tube to transition the catheter tube from the collapsed configuration to the expanded configuration.

18. The method according to claim 16, wherein in the collapsed configuration, a first portion of the inner surface of the catheter lumen contacts a second portion of the inner surface of the catheter lumen, the first portion being disposed opposite the second portion across a central axis of the catheter lumen.

19. The method according to claim 16, wherein the catheter tube further includes a reinforcement member extending longitudinally along the catheter tube.

20. The method according to claim 19, further including inflating the reinforcement member to transition the reinforcement member from a deflated state to an inflated state.

21. The method according to claim 20, wherein the reinforcement member in the inflated state imparts rigid mechanical properties on the catheter tube.

22. The method according to claim 20, wherein the reinforcement member in the inflated state transitions the catheter tube from a collapsed configuration to an expanded configuration.
